(19) United States
(12) Patent Application Publication
(10) Pub. No.: US 2007/0104743 A1
(43) Pub. Date: May 10, 2007

(54) Formulations of fispemifene

(75) Inventors: Veli-Matti Lehtola, Turku (FI); Kaija Halonen, Rusko (FI)

Correspondence Address: FITZPATRICK CELLA HARPER & SCINTO, 30 ROCKEFELLER PLAZA, NEW YORK, NY, 10112, US

(73) Assignee: HORMOS MEDICAL LTD., Turku (FI)

(21) Appl. No.: 11/594,891

(22) Filed: Nov. 9, 2006

Related U.S. Application Data
(60) Provisional application No. 60/734,935, filed on Nov. 9, 2005.

(51) Int. Cl. A61K 31/075; A61K 9/48; A61K 9/00

(52) U.S. Cl. 424/400; 424/451; 514/720

(57) ABSTRACT

This invention relates to a liquid or semisolid oral drug formulation comprising a therapeutically active compound of the formula (I) 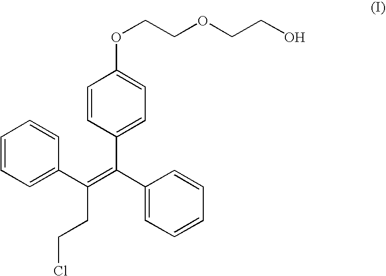 or a geometric isomer, a stereoisomer, a mixture of isomers, a pharmaceutically acceptable salt, an ester thereof or a metabolite thereof, in combination with a pharmaceutically acceptable carrier.

### BACKGROUND OF THE INVENTION

This application claims the benefit of priority of U.S. provisional application No. 60/734,935, filed on Nov. 9, 2005, incorporated herein by reference in its entirety.

### FIELD OF THE INVENTION

This invention relates to a liquid or semisolid oral drug formulation comprising fispemifene or a closely related compound as active ingredient.

### BACKGROUND OF THE INVENTION

The publications and other materials used herein to illuminate the background of the invention, and in particular, cases to provide additional details respecting the practice, are incorporated by reference.

Estrogens are increasingly used for the treatment of climacteric symptoms in women. Estrogens are shown to be beneficial also in the prevention of Alzheimer's disease (Henderson, 1997) and in the lowering of LDL-cholesterol values and thus preventing cardiovascular diseases (Grodstein & Stampfer, 1998). However, estrogen use increases the risk of uterine and breast cancers (Lobo, 1995). New therapies which would have the benefits of estrogens, but not the carcinogenic risks are requested.

Selective estrogen receptor modulators (SERMs) have been developed to fulfill these requirements (Macgregor & Jordan, 1998). Selective estrogen receptor modulators have both estrogen-like and antiestrogenic properties (Kauffman & Bryant, 1995). The effects may be tissue-specific as in the case of tamoxifen and toremifene which have estrogen-like effects in the bone, partial estrogen-like effect in the uterus and liver, and pure antiestrogenic effect in breast cancer. Raloxifene and droloxifen are similar to tamoxifen and toremifene, except that their antiestrogenic properties dominate. Based on the published information, many SERMs have important benefits in elderly women: they decrease total and LDL cholesterol, thus diminishing the risk of cardiovascular diseases, and they may prevent osteoporosis and inhibit breast cancer growth in postmenopausal women.

The US patents U.S. Pat. Nos. 6,576,645 and 6,875,775 describe a novel group of SERMs which are tissue-specific estrogens and which can be used in women in the treatment of climacteric symptoms, osteoporosis, Alzheimer's disease and/or cardiovascular diseases without the carcinogenic risk. Certain compounds can be given to men to protect them against osteoporosis, cardiovascular diseases and Alzheimer's disease without estrogenic adverse events (gynecomastia, decreased libido etc.). Of the compounds described in said patents, the compound (Z)-2-{2-[4-(4-chloro-1,2-diphenylbut-1-enyl)phenoxy]ethoxy}ethanol (also known under the generic name fispemifene) has shown a very interesting hormonal profile suggesting that it will be especially valuable for treating disorders in men, particularly for preventing osteoporosis in men. The published US patent application Publ. No. 2004-0248989 suggests the use of fispemifene for treatment or prevention of lower urinary tract symptoms such as detrusor urethral sphincter dyssynergia, abacterial prostatitis, stress prostatitis, trigonitis and orchialgia in male individuals, and interstitial cystitis in male or female individuals.

Fispemifene is the Z-isomer of the compound of formula (I)

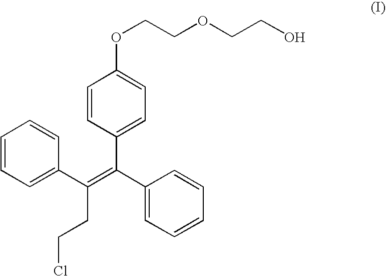

Fispemifene is only sparingly soluble in water.

### OBJECT AND SUMMARY OF THE INVENTION

An object of the present invention is to provide an improved drug formulation containing as active ingredient a compound of formula (I) or an isomer, especially fispemifene, or a mixture of isomers, a salt, ester or metabolite thereof, in which the dissolution and absorption of the active ingredient is essentially increased.

Thus, the invention concerns a liquid or semisolid oral drug formulation comprising a therapeutically active compound of the formula (I) 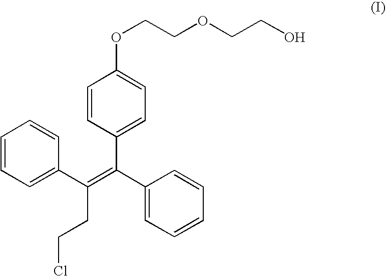
or a geometric isomer, a stereoisomer, a mixture of isomers, a pharmaceutically acceptable salt, an ester thereof or a metabolite thereof, in combination with a pharmaceutically acceptable carrier.

### BRIEF DESCRIPTION OF THE DRAWINGS

FIGS. 1 and 2 show individual serum concentration of fispemifene versus time in two female Cynomolgus monkeys ( #05084 and #06170, respectively) after administration of a single dose of 500 mg/kg of fispemifene in two different vehicles.

### DETAILED DESCRIPTION OF THE INVENTION

The term “liquid formulation” refers here particularly to a solution, a suspension with solid particles dispersed in a liquid, or a combination thereof, or an emulsion with liquid droplets dispersed in a liquid, or to a syrup. The “liquid” can be hydrophilic or lipophilic, preferably lipophilic.

The term “semisolid formulation” refers especially to gels and pastes.

According to one preferred embodiment, the liquid drug formulation is a solution of compound I or its isomer(s), salt, ester or metabolite in as suitable carrier, which can be a single carrier or a mixture of several carriers. The compounds of formula I have low solubility in water. The carrier shall therefore preferably comprise one or more lipophilic ingredients. In order to achieve enhanced bioavailability it is preferable to use digestible lipids such as triglycerides, diglycerides, fatty acids, phospholipids, or the like instead of indigestible oils such as mineral oils (Porter and Charman, 2001). A special group of useful carriers or ingredients therein may be cholane derivatives. U.S. Pat. No. 4,117,121 disclosed a group of cholane derivatives useful to decrease cholesterol level and to increase bile flow. A particularly preferred group of carriers is liquid fats (oils), especially vegetable oils such as corn oil, coconut oil or the like. The bioavailability enhancing ingredients and carriers are, however, not restricted to the aforementioned.

According to another preferred embodiment, the liquid drug formulation is a suspension of fine solid particles of the compound I in a liquid. The liquid can be a lipophilic or hydrophilic liquid or a mixture of several liquids. Said liquids can also comprise dissolved ingredients. By decreasing the particle size of the dispersed drug compound, the surface area available for digestion and drug release is enhanced. Preferably at least 90% of the drug substance shall have a particle size less than 150 micrometer, and 50% of the drug substance shall have a particle size less than 25 micrometer. Especially preferably, 90% of the drug substance shall have a particle size less than 50 micrometer, and 50% of the drug substance shall have a particle size less than 15 micrometer.

According to a third preferred embodiment, the liquid formulation is an emulsion. Because the aqueous solubility of compound I is very low, the emulsion is preferably a dispersion of a lipophilic phase (e.g., a solution and/or suspension of compound I in a lipophilic liquid) in an aqueous phase (oil-in-water emulsion). The emulsion may comprise additional components such as stabilizers (surfactants), emulsifiers and thickeners. According to a particularly preferred embodiment, the emulsion is a microemulsion or nanoemulsion. Micro- and nanoemulsions are, in contrast to conventional emulsions, isotropic, transparent and thermodynamically stable. The average size of the dispersed droplets is in a microemulsion typically about 10000 nm or below and in a nanoemulsion 100 nm or below.

According to a fourth preferred embodiment, the liquid formulation is a syrup.

Typical examples of semisolid oral formulations are gels and pastes. Gels are created by adding a gelatinizer such as gelatine or a polysaccharide to a solution, suspension or emulsion comprising compound I. According to one preferred embodiment, the gel is created by addition of a gelatinizer to a microemulsion according to EP 760651 B 1.

Although the liquid formulations such as solutions, emulsions and suspensions can be packed in larger bottles for many doses, it may be preferable to have the drug formulation packed into a unit dosage form, such as a capsule. Such capsule formulations are called softgel capsules. Soft gelatin capsules (or softgel capsules) consist of a liquid or semisolid matrix inside a one-piece outer shell, such as a gelatin shell. The drug compound itself may be either in solution, suspension or emulsion in the capsule-fill matrix. The characteristics of the fill matrix may be hydrophilic (for example polyethylene glycols) or lipophilic (such as triglyceride vegetable oils), or a mixture of both hydrophilic and lipophilic ingredients.

Significant advances have been made in recent years in the formulation of fill matrices. As examples can be mentioned microemulsions or nanoemulsions of the drug encapsulated as preconcentrates in the capsule. This means that the fill matrix is a concentrated micro- or nanoemulsion, i.e., a combination of a lipophilic liquid containing the hydrophobic drug, a small amount of hydrophilic liquid and a surfactant. After oral administration the microemulsion will become diluted in the gastrointestinal fluid. Alternatively, the matrix may comprise only the ingredients, i.e., the drug, a lipid or a lipid mixture and one or more surfactants. The ingredients will, upon administration, spontaneously create a microemulsion (or nanoemulsion) in the gastrointestinal fluid.

The softgel capsule consists for example of gelatin, water and a plasticizer. It may be transparent or opaque, and can be coloured and flavoured if desired. Preservatives are not required owing to the low water activity in the finished product. The softgel can be coated with enteric-resistant or delayed-release material. Although virtually any shape softgel can be made, oval or oblong shapes are usually selected for oral administration.

The term “metabolite” shall be understood to cover any fispemifene metabolite. One important metabolite is ospemifene or (deaminohydroxy)toremifene, which has the formula

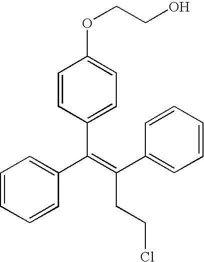

Other important fispemifene metabolites are the ospemifene metabolites 4-hydroxyospemifene, which has the formula 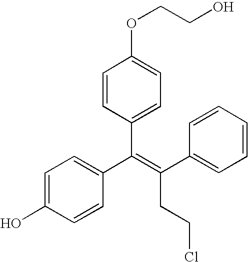
and the corresponding 3-hydroxyospemifine. Further examples of metabolites are the toremefine metabolites mentioned in Kangas (1990) on page 9: 4-hydroxy (deaminohydroxy) toremifene (TORE VI), 4,4′-dihydroxy(deaminohydroxy) toremifene (TORE VII), deaminocarboxy toremifene (TORE XVIII), ); 4-hydroxy(deaminocarboxy) toremifene (TORE VIII), and toremifene monophenol (TORE XIII); especially TORE VI and TORE XVIII.

The compound (I) is preferably the Z-isomer, i.e., fispemifene.

The improved drug formulation according to this invention is useful in any application of fispemifene, especially for use in treatment or prevention of osteoporosis, cardiovascular diseases, Alzheimer disease, lower urinary tract symptoms, or for treatment or prevention of prostate cancer in men.

The required dosage of compound (I) in the formulation according to this invention will vary with the particular condition being treated or prevented, the severity of the condition, and the specific carrier employed. The optimal clinical dose of fispemifene is expected to be higher than 5 mg daily and lower than 300 mg daily. A particularly preferable daily dose has been suggested in the range 20 to 200 mg. Due to the enhanced bioavailability according to the method of this invention, it can be predicted that the same therapeutic effect can be achieved with doses lower those estimated earlier.

The invention will be disclosed more in detail in the following non-restrictive Example.

### EXAMPLE

Serum concentration of fispemifene in monkeys after administration of fispemifene in two different vehicles

A pilot study on exposure of fispemifene in two female Cynomolgus monkeys ( #05084 and #06170) was carried out. Fispemifene was administered by single oral dosing of 500 mg/kg in two different vehicles, 0.5% carboxymethyl cellulose in water (CMC), and in corn oil. Blood samples were collected 0, 1, 2, 4, 6, 8, 12, 16 and 24 hours after dosing. Concentrations of fispemifene were determined using LC-MS/MS.

Results:

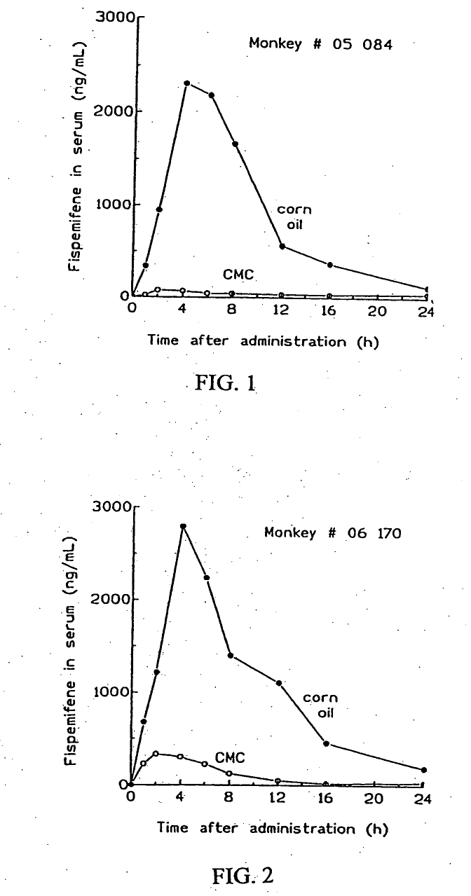

Fispemifene was quantifiable in all serum samples taken after drug administration. Individual serum fispemifene concentrations versus time for the two monkeys are shown in FIGS. 1 and 2. It can be seen that serum fispemifene concentration is more than 10-fold higher from corn oil vehicle than from 0.5 CMC in aquoeous solution. This experiment shows that a lipophilic liquid such as an oil is an excellent carrier for dissolution and/or suspension of fispemifene.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

What is claimed is:

1. A liquid or semisolid oral drug formulation comprising a therapeutically active compound of the formula (I) 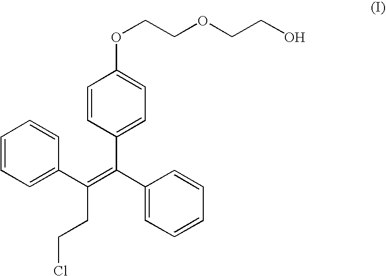
or a geometric isomer, a stereoisomer, a mixture of isomers, a pharmaceutically acceptable salt, an ester thereof or a metabolite thereof, in combination with a pharmaceutically acceptable carrier.

2. The drug formulation according to claim 1 wherein compound (I) is fispemifene.

3. The drug formulation according to claim 1 wherein said formulation is selected from the group consisting of a solution, a suspension and a combination of a solution and suspension.

4. The drug formulation according to claim 1 wherein said therapeutically active compound is dissolved and/or suspended in an oil.

5. The drug formulation according to claim 1 wherein said formulation is an emulsion.

6. The drug formulation according to claim 5 wherein the emulsion is a microemulsion or nanoemulsion.

7. The drug formulation according to claim 1 wherein the formulation is a syrup.

8. The drug formulation according to claim 1 wherein the formulation is a gel.

9. The drug formulation according to claim 1 wherein the formulation is a paste.

10. The drug formulation according to claim 1 wherein said formulation is packed into a unit dosage form.

11. The drug formulation according to claim 10 wherein the dosage form is the formulation encapsulated in a soft capsule.

12. The drug formulation according to claim 1 wherein the carrier comprises a bile flow promoting agent.
